(19) United States
(12) Patent Application Publication
(10) Pub. No.: US 2024/0127939 A1
(43) Pub. Date: Apr. 18, 2024

(54) METHODS AND SYSTEMS FOR PARAMETER MODELING OF SIMULATED PATIENT FLOW

(71) Applicant: KONINKLIJKE PHILIPS N.V., Eindhoven (NL)

(72) Inventors: Lasith Adhikari, Cambridge, MA (US); David Paul Noren, Cambridge, MA (US); Gregory Boverman, Cambridge, MA (US); Eran Simhon, Cambridge, MA (US); Chaitanya Kulkarni, Bangalore (IN); Moumita Saha, Bangalore (IN); Krishnamoorthy Palanisamy, Bangalore (IN); Gyana Ranjan Mallick, Bangalore (IN); Ahmed Sanin, Bangalore (IN); Claire Yunzhu Zhao, Cambridge, MA (US)

(21) Appl. No.: 18/381,236

(22) Filed: Oct. 18, 2023

Related U.S. Application Data
(60) Provisional application No. 63/417,065, filed on Oct. 18, 2022.

(51) Int. Cl. G16H 40/20; G16H 10/60

(52) U.S. Cl. CPC G16H 40/20; G16H 10/60

(57) ABSTRACT

A method for predicting simulated patient admissions, comprising: receiving healthcare records for a plurality of patients; adapting the received healthcare records to a common data format; parameterizing the adapted healthcare records to generate a plurality of patient parameters comprising for each patient a day of the week admission parameter, a time of day admission parameter, and a patient type parameter; generating a length of stay parameter for each of the plurality of different patient types; generating a transition probability for each of the plurality of different patient types; predicting, for a time period in the healthcare environment, patient admissions; predicting a care pathway for some or all of the predicted patient admissions during the time period; and reporting, via a user interface, the predicted patient admissions and predicted care pathways.

### FIELD OF THE INVENTION

The present disclosure is directed generally to methods and systems for predicting simulated patient admissions and patient care pathways for a healthcare environment.

### BACKGROUND

Accurately predicting resource utilization, including human resources, bed space, and medical resources, is particularly important in the healthcare setting. Optimizing the utilization of bed space, for example, is a high priority for hospital management in order to avoid bottleneck situations. Similarly, understanding or predicting the expected future utilization of human resources and medical resources can avoid both strain on and underutilization of these resources.

Accordingly, simulation-based patient flow modeling is increasingly used as an operational decision support tool in modern-day hospitals. These patient flow simulation models help in understanding potential future bottlenecks in staffing and resource utilization by forecasting hospital census. Regardless of which technology is used in simulation models, there are two main types of patients that need to be modeled: (1) existing patients at the time of running the simulation (i.e., the current time point), and (2) virtual patients who could arrive at the hospital after the current time point (i.e., “simulated patients”).

However, exiting patient flow simulation models are limited to modeling patient flow using only straightforward historical admission/discharge/transfer data. For example, on an average Friday between 1 PM and 3 PM there are 25 patients admitted and 20 patients discharged. These simplistic models typically leverage clinical information only for very small and specific populations, such as acute coronary syndrome, and are limited to certain parameters, such as clinical pathways. Accordingly, since electronic healthcare records and medical records are not available for future arrivals at the time of running the simulation, there are no sufficiently accurate models that utilize clinical data specifically to model simulated patients across a healthcare environment.

### SUMMARY OF THE INVENTION

Accordingly, there is a continued need for systems and methods that more accurately predict future patient admissions for a healthcare environment.

The present disclosure is directed to inventive methods and systems for predicting simulated patient admissions and patient care pathways for a healthcare environment. A patient prediction system receives healthcare records for a plurality of patients, comprising a plurality of different patient types. The system adapts the healthcare records to a common data format, and parameterizes the adapted healthcare records to generate a plurality of patient parameters, including a day of the week admission parameter, a time of day admission parameter, and a patient type parameter. The system also uses the healthcare records to generate a length of stay parameter for each of the plurality of different patient types, and to generate a transition probability for each of the different patient types. The system predicts patient admissions using the patient parameters, and predicts a care pathway for some or all of the predicted patient admissions using the length of stay parameters and the transition probabilities. Some or all of this information can be provided to a user or another system via a user interface.

Generally in one aspect, a method for predicting simulated patient admissions and patient care pathways for a healthcare environment is provided. The method includes: (i) receiving, from an electronic health record database, healthcare records for a plurality of patients, wherein the plurality of patients comprises a plurality of different patient types; (ii) adapting the received healthcare records to a common data format; (iii) parameterizing the adapted healthcare records to generate a plurality of patient parameters, the plurality of patient parameters comprising for each patient a day of the week admission parameter, a time of day admission parameter, and a patient type parameter; (iv) generating, from the plurality of patient parameters, a length of stay parameter for each of the plurality of different patient types; (v) generating, from the plurality of patient parameters, a transition probability for each of the plurality of different patient types, wherein the transition probabilities are stratified by one or more of likely day of transition and likely hour of transition; (vi) predicting, for a time period in the healthcare environment, patient admissions using the patient parameters, wherein the patient admissions comprises predicted numbers of admitted patients during the time period and predicted patient type for each of the admitted patients; (vii) predicting, using the length of stay parameters and the transition probabilities, a care pathway for some or all of the predicted patient admissions during the time period; and (viii) reporting, via a user interface, the predicted patient admissions and predicted care pathways.

According to an embodiment, the predicted care pathway comprises a length of stay prediction and one or more predicted transitions for a patient.

According to an embodiment, the method further comprises applying a dimensionality reduction to the patient type parameters. According to an embodiment, the dimensionality reduction comprises clustering the patient type parameters by a similarity factor. According to an embodiment, the dimensionality reduction comprises clustering the patient type parameters by ICD codes in the healthcare records.

According to an embodiment, the patient type parameter comprises one or more of patient admission diagnosis, patient discharge diagnosis, patient reason for visit, and patient complaint.

According to an embodiment, the method further comprises repeating the step of predicting a care pathway for some or all of the predicted patient admissions for one or more subsequent time periods.

According to an embodiment, reporting the predicted patient admissions and predicted care pathways comprises reporting a patient census for the healthcare environment for one or more time periods.

According to another aspect is a system for predicting simulated patient admissions and patient care pathways for a healthcare environment. The system includes: an electronic health record database comprising healthcare records for a plurality of patients, wherein the plurality of patients comprises a plurality of different patient types; a processor configured to: (i) adapt the received healthcare records to a common data format; (ii) parameterize the adapted healthcare records to generate a plurality of patient parameters, the plurality of patient parameters comprising for each patient a day of the week admission parameter, a time of day admission parameter, and a patient type parameter; (iii) generate, from the plurality of patient parameters, a length of stay parameter for each of the plurality of different patient types; (iv) generate, from the plurality of patient parameters, a transition probability for each of the plurality of different patient types, wherein the transition probabilities are stratified by one or more of likely day of transition and likely hour of transition; (v) predict, for a time period in the healthcare environment, patient admissions using the patient parameters, wherein the patient admissions comprises predicted numbers of admitted patients during the time period and predicted patient type for each of the admitted patients; and (vi) predict, using the length of stay parameters and the transition probabilities, a care pathway for some or all of the predicted patient admissions during the time period; and a user interface configured to report the predicted patient admissions and predicted care pathways.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

These and other aspects of the invention will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 is a flowchart of a method for predicting simulated patient admissions and patient care pathways for a healthcare environment, in accordance with an embodiment.

FIG. 2 is a schematic representation of a patient prediction system, in accordance with an embodiment.

FIG. 3 is a schematic representation of patient tracking and prediction methods, in accordance with an embodiment.

FIG. 4 is a schematic representation of a patient prediction system, in accordance with an embodiment.

FIG. 5 is a schematic representation of a method of prediction by a patient prediction system, in accordance with an embodiment.

FIG. 6 is a schematic representation of a method of prediction by a patient prediction system, in accordance with an embodiment.

### DETAILED DESCRIPTION OF EMBODIMENTS

The present disclosure describes various embodiments of a method and system for predicting simulated patient admissions and patient care pathways for a healthcare environment. More generally, Applicant has recognized and appreciated that it would be beneficial to provide an improved system that can more accurately predict future patient admissions. Accordingly, a patient prediction system receives healthcare records for a plurality of patients, comprising a plurality of different patient types. The system adapts the healthcare records to a common data format, and parameterizes the adapted healthcare records to generate a plurality of patient parameters, including a day of the week admission parameter, a time of day admission parameter, and a patient type parameter. The system also uses the healthcare records to generate a length of stay parameter for each of the plurality of different patient types, and to generate a transition probability for each of the different patient types. The system predicts patient admissions using the patient parameters, and predicts a care pathway for some or all of the predicted patient admissions using the length of stay parameters and the transition probabilities. Some or all of this information can be provided to a user or another system via a user interface.

According to an embodiment, the systems and methods described or otherwise envisioned herein can, in some non-limiting embodiments, be implemented as an element for a commercial product for patient analysis, monitoring, or management, such as the Philips® Patient Flow Capacity Suite (PFCS) (available from Koninklijke Philips NV, the Netherlands), or any suitable system. For example, the system and method can be implemented within existing or future clinical decision support systems (CDS), applications, and devices. However, the disclosure is not limited to these devices or systems, and thus disclosure and embodiments disclosed herein can encompass any device or system capable of predicting future patient admissions or utilizing such a prediction.

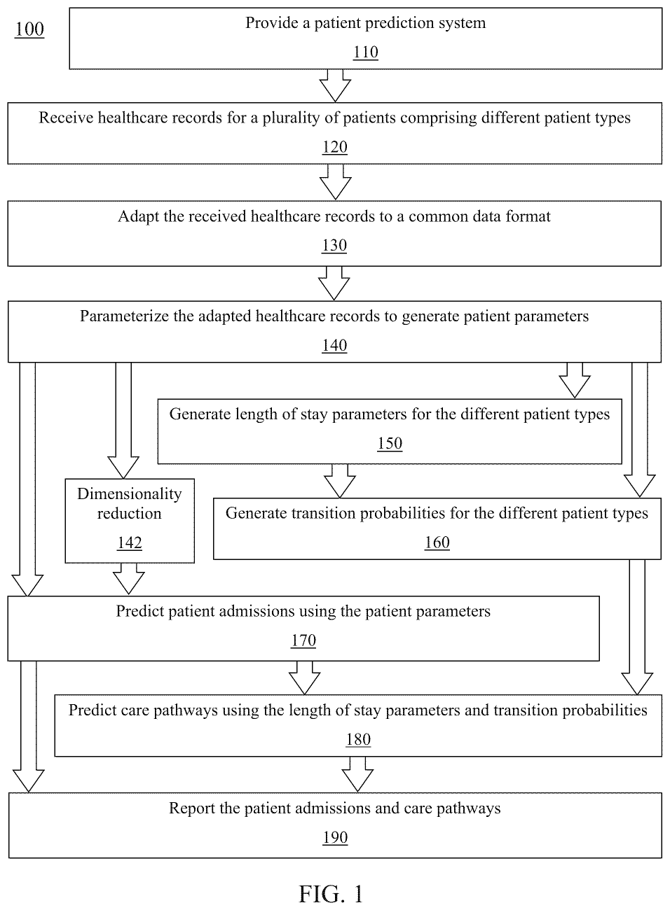

Referring to FIG. 1, in one embodiment, is a flowchart of a method 100 for developing a set of consensus criteria for patient analysis using a patient prediction system. The methods described in connection with the figures are provided as examples only, and shall be understood not to limit the scope of the disclosure. The patient prediction system can be any of the systems described or otherwise envisioned herein. The patient prediction system can be a single system or multiple different systems.

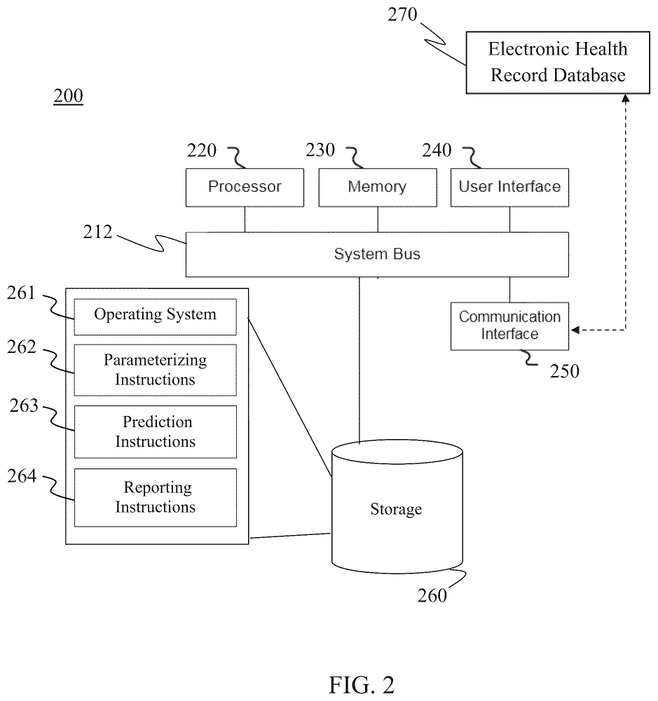

At step 110 of the method, a patient prediction system is provided. Referring to an embodiment of a patient prediction system 200 as depicted in FIG. 2, for example, the system comprises one or more of a processor 220, memory 230, user interface 240, communications interface 250, and storage 260, interconnected via one or more system buses 212. It will be understood that FIG. 2 constitutes, in some respects, an abstraction and that the actual organization of the components of the system 200 may be different and more complex than illustrated. Additionally, patient prediction system 200 can be any of the systems described or otherwise envisioned herein. Other elements and components of the patient prediction system 200 are disclosed and/or envisioned elsewhere herein.

According to an embodiment, the patient prediction system comprises or is in communication with an Electronic Health Record (EHR) system or service 270. The EHR system or service 270 can be, for example, a database of patient data, health information, medical records, and any other information for one or more patients. Thus, the EHR database can be any database comprising patient data for one or more patients or subjects. The EHR system or service 270 may be a local or remote system and can be in direct and/or indirect communication with the patient data display system. The patient data, health information, medical records and any other information stored in the EHR database can comprise any data about a patient. For example, the patient records may comprise demographic information, admission information including day and time of admission, diagnostic information, clinical measurements, treatment information, vital record data, transition information, discharge information, and/or any other information, including over a period of time for each of the plurality of patients. Thus, the EHR system or service can be, for example, a recipient and/or source of past and current medical data for a patient, such current patient monitoring data, vital sign data, and much more.

According to an embodiment, the patient prediction system is a scalable framework that can assist with leveraging hospital specific clinical insights to simulated patients. The single system provides clinically informed dynamic parameter predictions for potential future arrivals, and can be remodeled at any desired frequency in order to provide adaptivity. It further provides a simulated patient-specific iterative approach to form more realistic unit trajectories along with a unit and time specific length of stay values.

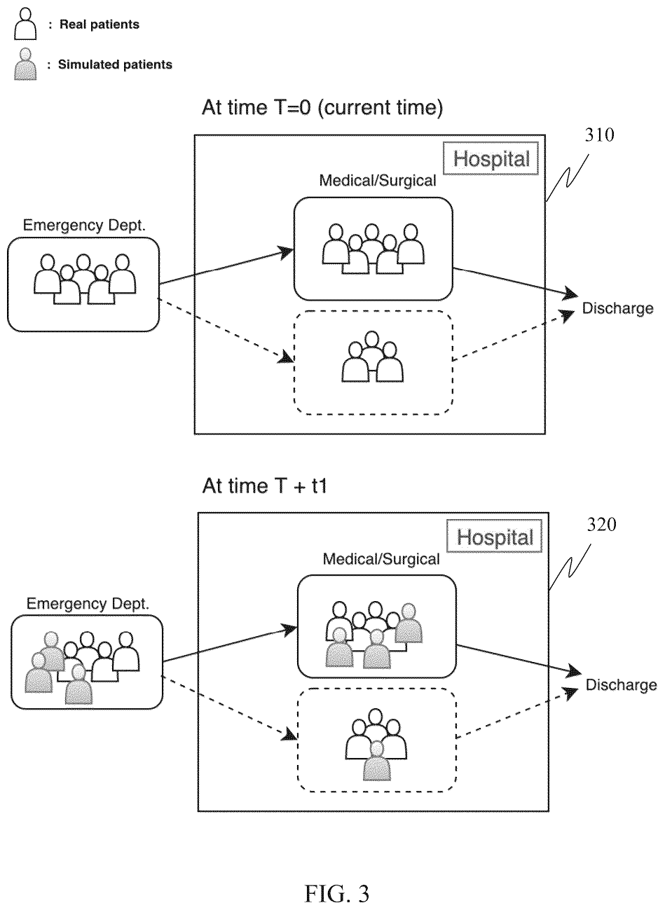

To improve the accuracy of unit level census predictions for the ensuing 24-72 hours (or any other future time period), it is critical to model the input parameters (e.g., unit trajectory, unit-level length of stay, etc.) of simulated patients based on their predicted clinical background. Referring to FIG. 3, in accordance with an embodiment, is a graphic depicting the two types of patients that must be modeled, along with the situations for which each can be utilized. So-called “real patients” are existing patients at the healthcare setting at the time of running a simulation or prediction (i.e., the current time point). So-called “simulated patients” are future patients that are predicted to arrive at the healthcare setting after the current time point. In 310, at time T=0 (i.e., the current time), the healthcare setting comprises only admitted patients in their wards (i.e., the ‘real patients’). In 320, at T+t1, there are both the current “real” patients and the newly arrived patients (i.e., “simulated patients”).

At step 120 of the method, the patient prediction system 200 receives, retrieves, or otherwise obtains healthcare records for a plurality of patients, wherein the plurality of patients comprises a plurality of different patient types. The healthcare records can comprise any information about the patients that may be utilized by the patient prediction system as described or otherwise envisioned herein. For example, the patient records may comprise demographic information, admission information including day and time of admission, diagnostic information, clinical measurements, treatment information, vital record data, transition information, discharge information, and/or any other information.

According to an embodiment, a patient type may be anything that can be used to cluster, group, classify, stratify, or otherwise identify multiple patients into a group. For example, a patient type may be defined by or based on admitting diagnosis, working or final diagnosis, reason for visit or admission, patient complaints, and many other options. A patient type may also be defined by, for example, an injury or disease identification, such as an ICD code used for the patient (where ICD is the International Classification of Diseases maintained by the World Health Organization (WHO)). Other patient type definitions or identifiers are possible.

According to an embodiment, the plurality of patients are patients of the healthcare setting or another healthcare setting during a period of time before the retrieval of the healthcare records. The period of time can be any period that is suitable to result in records that can be used by, and are sufficient for acceptable results generated by, patient prediction system 200. For example, the period of time may be a week, a month, six months, a year, multiple years, or any other time period less than, in between, or greater than these example period of times. As just one possible embodiment, the patient prediction system can receive or obtain healthcare records for a plurality of patients of a plurality of different patient types for a period of one year leading up to the time point of prediction.

According to an embodiment, the patient healthcare records can be received, retrieved, or otherwise obtained by the patient prediction system 200 from any source. The source may be the EHR system or database 270, a clinical decision support (CDS) system, or any other system or source. The patient healthcare records can be continually or periodically fed into or requested by the patient prediction system. The patient healthcare records may be temporarily stored in a local or remote database before being utilized by the patient prediction system.

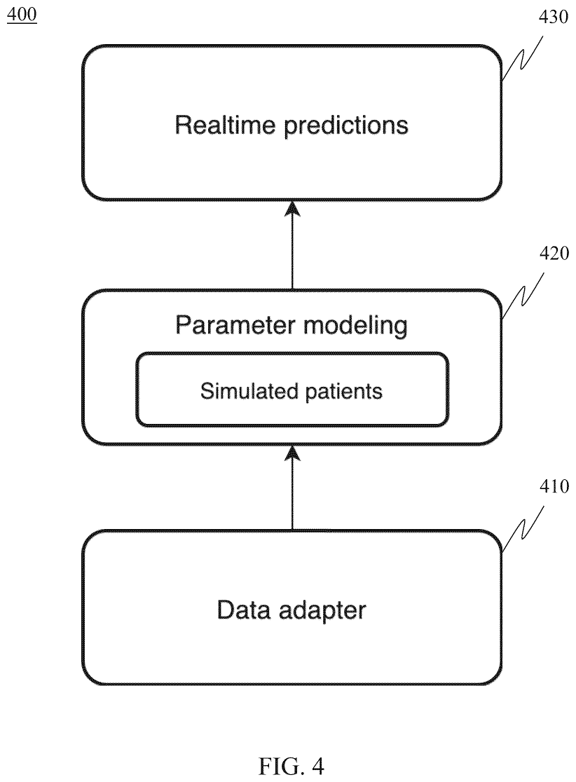

At step 130 of the method, the patient prediction system adapts or modifies or filters or otherwise converts the received healthcare records to a common data format. The common data format can be any format that can be utilized by the patient prediction system. Referring to FIG. 4, in one embodiment, is a high-level schematic representation 400 of a patient prediction system with the main elements including a data adapter 410. The data adapter is a module that reads all the received electronic healthcare records (e.g., sociodemographic, diagnosis, comorbidities, clinical events, historical hospital visit data) into a common data format, including following a predefined pre-processing pipeline. This module also enables the system to easily interface the proposed prediction models with the IT (information technology) systems of different healthcare settings with minimal changes to the overall system. In other words, since the data adapter can be configured to recognize and utilize healthcare records of any format, either based on existing programming or based on user input or modification of programming, and since the data adapter can convert those healthcare records into a single common data format that can be utilized in downstream steps of the patient prediction analysis, the patient prediction system can be implemented in a variety of different healthcare settings with minimal adaption.

According to an embodiment, once the received healthcare records are converted or modified or adapted to a common data format, those healthcare records may be utilized immediately or may be stored in a local or remote database before being utilized by the patient prediction system.

At step 140 of the method, the adapted healthcare records are parameterized to generate a plurality of patient parameters. A healthcare record can be parameterized using any method for data parameterization. A plurality of healthcare records associated with a single patient can all be parameterized and associated with that single patient. According to an embodiment, the plurality of patient parameters for each patient can include a day of the week admission parameter, a time of day admission parameter, and a patient type parameter, although other parameters are possible. For example, the patient parameters for a patient X1 may comprise the day of the week admission parameter (Tuesday), a time of day admission parameter (1132), and a patient type parameter (broken right tibia). The patient parameters for a patient X2 may comprise the day of the week admission parameter (Saturday), a time of day admission parameter (1904), and a patient type parameter (fever).

Referring again to FIG. 4, in one embodiment, is a high-level schematic representation 400 of a patient prediction system with the main elements including a parameter modeling module 420. The parameter modeling module receives the patient healthcare records in the common data format from the data adapter module (or from memory) and performs the parameter modeling required by the simulation. According to an embodiment, the system models parameters for simulated patients including length of stay and clinical pathway as described below. According to an embodiment, the clinically informed parameter modeling approach can be specific to a given hospital/institute and can be adaptive over the time. As an example, the modelling approach can be triggered daily, weekly, monthly, or at other time periods to remodel parameters to learn from more recent changes.

According to an embodiment, once the received healthcare records are parameterized, those parameters may be utilized immediately or may be stored in a local or remote database before being utilized by the patient prediction system.

At optional step 142 of the method, the patient prediction system can perform a dimensionality reduction of the patient type parameters, and/or to any other identified parameter. For example, the system can use a clustering method or use the category of ICD codes to identify the general type of the injury or disease, and can group or cluster patients and/or patient types into a smaller number of groups via the similarities or ICD codes. Many algorithms and methods for dimensionality reduction are known, or are possible.

Following step 140 (or optionally step 142), the patient prediction system 200 comprises a set of parameters that enables the system to generate real-time predictions of simulated patients and patient trajectories. Referring again to FIG. 4, in accordance with an embodiment, is the high-level schematic representation 400 of the patient prediction system. After parameterization (and optionally dimensionality reduction) by the parameter modeling module 420, the resulting parameter set—which can be utilized immediately or stored for downstream or subsequent use—can be used by a realtime prediction module 430 to generate real-time predictions of simulated patients and patient trajectories. According to an embodiment, the realtime prediction module 430 reads generated parameters (e.g., from stored j son files) from the parameter modeling module and performs real time predictions of simulated patient trajectories including their estimated length of stays. To achieve that, first the most likely patient types will be assigned to predicted simulated patients and then the system will generate trajectories for those simulated patients using iterative approach as explained below.

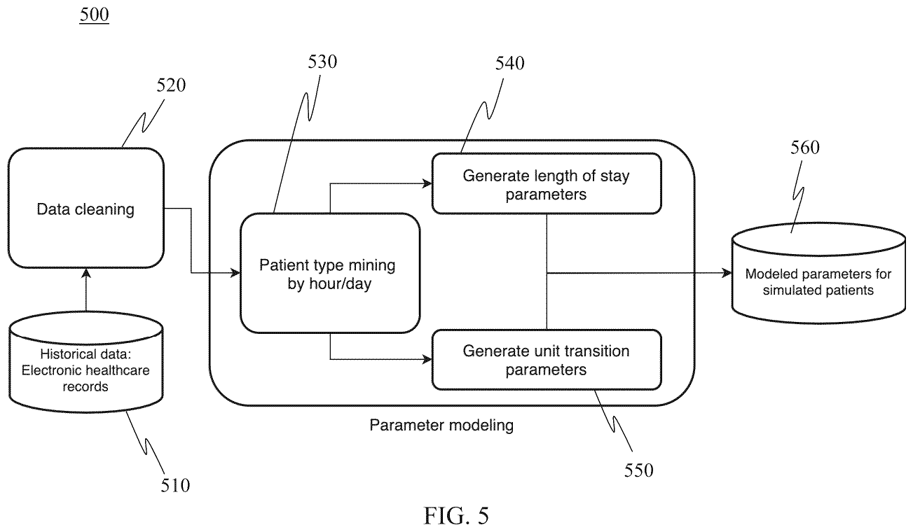

Referring to FIG. 5, in one embodiment, is a high-level schematic representation 500 of the method of prediction by the patient prediction system. The system begins at 510 with healthcare records for a plurality of patients. At 520, the healthcare records are processed including cleaning, filtering, and converting to a common data format. The processed healthcare records can then be used for parameter modeling. At 530, the processed healthcare records are parameterized to generate a plurality of patient parameters including a day of the week admission parameter, a time of day admission parameter, and a patient type parameter. Thus, patient types are associated with admission days and admission times, in accordance with an embodiment.

At step 150 of the method, the patient prediction system uses the plurality of patient parameters to generate a length of stay parameter for each of the plurality of different patient types. This can be accomplished according to a variety of different mechanisms. Referring again to FIG. 5, in one embodiment, is a high-level schematic representation 500 of the method of prediction by the patient prediction system. At 540, the patient prediction system computes length of stay parameters for each identified patient type. According to an embodiment, the system can account for the patient admit hour and potential demographics (e.g., known age distributions, known sociodemographic factors from historical data, etc.) along with the patient type. According to an embodiment, the length of stay parameters can be computed using a statistical/machine learning approach (e.g., fitting time to event model) or by generating inverted empirical cumulative functions for each patient type. The generated length of stay parameters can be stored for real time use.

At step 160 of the method, the patient prediction system uses the plurality of patient parameters to generate a transition probability for each of the plurality of different patient types. According to an embodiment, the transition probabilities can be stratified by one or more of likely day of transition and likely hour of transition. This can be accomplished according to a variety of different mechanisms. Referring again to FIG. 5, in one embodiment, is a high-level schematic representation 500 of the method of prediction by the patient prediction system. At 550, the patient prediction system computes these unit transition probabilities. According to an embodiment, in order to construct clinical pathways for simulated patients, transition probabilities are generated for each identified main patient type. In particular, the system can stratify these transition probabilities by the hour and day of the transition. The generated unit transition parameters can be stored for real time use.

Thus, at 560, the patient prediction system comprises a plurality of patient parameters, generated length of stay parameters, and generated unit transition parameters (or probabilities), which it can utilize for downstream analysis as described or otherwise envisioned herein. For example, the stored parameters can be utilized in real time inside a simulation engine to predict the patient flow for virtual patients. According to an embodiment, each or all of these sets of parameters can be updated on a regular basis including daily, weekly, monthly, upon request, or at any other interval or upon any trigger or request. According to an embodiment, in order to perform clinically-informed parameter modeling, the system may use historical electronic healthcare records (EHR) from a target healthcare setting for at least the previous 1-2 years, although longer and shorter periods are possible.

At step 170 of the method, the patient prediction system predicts patient admissions using the patient parameters, for a particular time period. According to an embodiment, the patient admissions comprise predicted numbers of admitted patients during the particular time period and predicted patient type for each of the admitted patients. The particular time period may be a day and hour, a stretch of time such as an hour or a day, or any other time period. Thus, once the predicted number of patient arrivals for each hour are available (e.g., this can be obtained using the existing time series predictions or a Poisson arrival process) they can be mapped to identified patient types. This patient type assignment can be varied based on the hour of the day and the day of the week.

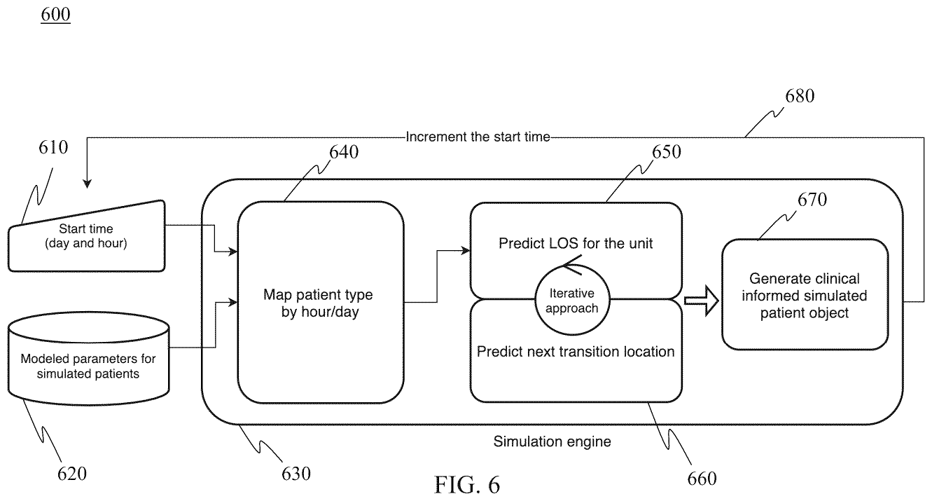

Referring to FIG. 6, in one embodiment, is a high-level schematic representation 600 of a method of prediction by the patient prediction system 200. At 640, the system predicts (“maps”) patients using an identified start hour and day (or any other time period) 610 using the stored modeled parameters for simulated patients 620 (which can include the plurality of patient parameters, generated length of stay parameters, and generated unit transition parameters (or probabilities) from 560 of the method depicted in FIG. 5 and described above). The identified start time 610 can be provided by a user, triggered by an event, or preprogrammed.

Accordingly, following step 170 of the method, the patient prediction system comprises information about a number of patient arrivals with patient type for a given start time. Now that the system has this information, it can generate a most likely care pathway including the unit length of stay values, using an iterative approach.

Accordingly, at step 180 of the method, the patient prediction system predicts, using the length of stay parameters and the transition probabilities, a care pathway for some or all of the predicted patient admissions.

According to an embodiment, the system uses the number of patient arrivals with patient type for a given start time (such as from 640 in FIG. 6) and the stored length of stay parameters (such as from 560 of FIGS. 5 and 620 of FIG. 6) to generate a predicted length of stay for the predicted patient admissions. According to an embodiment, at 650 the patient prediction system assigns unit length of stay for the entry unit (e.g., the emergency department, inpatient unit, etc.) using the stored clinically informed length of stay parameters from the modeling process.

Similarly, according to an embodiment, the system uses the number of patient arrivals with patient type for a given start time (such as from 640 in FIG. 6), the stored length of stay parameters (such as from 560 of FIGS. 5 and 620 of FIG. 6), and the stored transition probabilities (such as from 560 of FIGS. 5 and 620 of FIG. 6) to generate transition information. According to an embodiment, at 660 given the unit length of stay from above, the unit exit time (clock hour) is computed. Given the exit hour and the stored clinically informed parameters, the most likely next transition location (e.g., inpatient unit, discharge, etc.) can be selected. This can be an iterative approach.

Together, the predicted length of stay for the predicted patient admissions and the generated transition information comprises care pathways for the predicted patient admissions. Accordingly, at 670, the patient prediction system comprises care pathways for the predicted patient admissions (“clinical informed simulated patient objects”) which can also be thought of as patient trajectories comprising admission information (i.e., day and time of admission), length of stay, and one or more transitions. The care pathway or patient trajectory can also comprise a time of discharge, and other information.

According to an embodiment, the start time is incremented 680, and the process is repeated. The increment can be any increment of time, and may depend on the needs of the particular healthcare setting. For example, according to one embodiment, the increment is one minute, five minutes, ten minutes, thirty minutes, an hour, or any other time period shorter or longer than these example time periods. There can be a balance, for example, between the most accurate prediction (such as by incrementing by second(s)) and the computing power needed to perform these predictions.

Thus, following these predictions, the system comprises a plurality of predicted patient trajectories each comprising at least an admission date/time, transitions (if any), and discharge (if any), for a period of time. According to an embodiment, the patient trajectories generated from this iterative approach are used inside the simulation engine to extract a census (e.g., occupancy, hospital discharge counts, inpatient admission counts) in each clock tick.

At step 190 of the method, the patient prediction system reports the predicted patient admissions and predicted care pathways. The report can comprise information about the plurality of predicted patient trajectories each comprising at least an admission date/time, transitions (if any), and discharge (if any), for a period of time. The report can be provided using any of a variety of different mechanisms, and may depend on a request for information from a user. According to an embodiment, the report may comprise a patient count for a given time period or time point. For example, the report could comprise a graph or counter showing patient counts, such as patient admissions for a given time period or time point, or patient counts for a given unit or department of the healthcare setting for a given time period or time point, discharges for a given time period or time point, and a variety of other possible information. The report could comprise an overlay of the healthcare setting at one or more time points with patient counts. There are many other possible variations of the report.

Referring to FIG. 2 is a schematic representation of a patient prediction system 200. System 200 may be any of the systems described or otherwise envisioned herein, and may comprise any of the components described or otherwise envisioned herein. It will be understood that FIG. 2 constitutes, in some respects, an abstraction and that the actual organization of the components of the system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, the electronic medical record database 270 is a database from which the information about a plurality of patients, including demographic, diagnosis, and/or treatment information may be obtained or received. According to an embodiment, the electronic medical record database 270 is a database from which the patient parameters will be extracted. The electronic medical records database may be a local or remote database and is in direct and/or indirect communication with system 200. Thus, according to an embodiment, the system comprises an electronic medical record database or system 270.

According to an embodiment, storage 260 of system 200 may store one or more algorithms, modules, and/or instructions to carry out one or more functions or steps of the methods described or otherwise envisioned herein. For example, the system may comprise, among other instructions or data, parameterization instructions 262, prediction instructions 263, and/or reporting instructions 264.

According to an embodiment, parameterization instructions 262 direct the system to parameterize a plurality of received healthcare records to generate a plurality of patient parameters. A healthcare record can be parameterized using any method for data parameterization. A plurality of healthcare records associated with a single patient can all be parameterized and associated with that single patient. According to an embodiment, the plurality of patient parameters for each patient can include a day of the week admission parameter, a time of day admission parameter, and a patient type parameter, although other parameters are possible.

According to an embodiment, parameterization instructions 262 also direct the system to generate a length of stay parameter for each of the plurality of different patient types. This can be accomplished according to a variety of different mechanisms. According to an embodiment, the system can account for the patient admit hour and potential demographics (e.g., known age distributions, known sociodemographic factors from historical data, etc.) along with the patient type. According to an embodiment, the length of stay parameters can be computed using a statistical/machine learning approach (e.g., fitting time to event model) or by generating inverted empirical cumulative functions for each patient type. The generated length of stay parameters can be stored for real time use. The parameterization instructions 262 also direct the system to generate a transition probability for each of the plurality of different patient types. According to an embodiment, the transition probabilities can be stratified by one or more of likely day of transition and likely hour of transition. This can be accomplished according to a variety of different mechanisms. According to an embodiment, in order to construct clinical pathways for simulated patients, transition probabilities are generated for each identified main patient type. In particular, the system can stratify these transition probabilities by the hour and day of the transition. The generated unit transition parameters can be stored for real time use.

According to an embodiment, prediction instructions 263 direct the system to predict patient admissions using the patient parameters, for a particular time period. According to an embodiment, the patient admissions comprise predicted numbers of admitted patients during the particular time period and predicted patient type for each of the admitted patients. The particular time period may be a day and hour, a stretch of time such as an hour or a day, or any other time period. Thus, once the predicted number of patient arrivals for each hour are available (e.g., this can be obtained using the existing time series predictions or a Poisson arrival process) they can be mapped to identified patient types. This patient type assignment can be varied based on the hour of the day and the day of the week. According to an embodiment, prediction instructions 263 also direct the system to predict, using the length of stay parameters and the transition probabilities, a care pathway for some or all of the predicted patient admissions.

According to an embodiment, the reporting instructions 264 direct the system to generate and provide to a user via a user interface information comprising the predicted patient admissions and predicted care pathways. The report can comprise information about the plurality of predicted patient trajectories each comprising at least an admission date/time, transitions (if any), and discharge (if any), for a period of time. The report can be provided using any of a variety of different mechanisms, and may depend on a request for information from a user. According to an embodiment, the report may comprise a patient count for a given time period or time point. For example, the report could comprise a graph or counter showing patient counts, such as patient admissions for a given time period or time point, or patient counts for a given unit or department of the healthcare setting for a given time period or time point, discharges for a given time period or time point, and a variety of other possible information. The report could comprise an overlay of the healthcare setting at one or more time points with patient counts. There are many other possible variations of the report. The information may be communicated by wired and/or wireless communication to another device. For example, the system may communicate the information to a mobile phone, computer, laptop, wearable device, and/or any other device configured to allow display and/or other communication of the information.

According to an embodiment, the patient prediction system is configured to process many thousands or millions of datapoints in the input data used to normalize and parameterize the received patient records and information, and then to generate the described patient admission and care pathway predictions. These algorithms process millions of datapoints from input data, which require millions or billions of calculations. Thus, generating a patient prediction report comprises a process with a volume of calculation and analysis that a human brain cannot accomplish in a lifetime, or multiple lifetimes.

In addition, the patient prediction system can be configured to continually receive patient data, perform the analysis, and provide periodic or continual updates via the report provided to a user for the healthcare setting. This requires the analysis of thousands or millions of datapoints on a continual basis to optimize the reporting, requiring a volume of calculation and analysis that a human brain cannot accomplish in a lifetime. By providing an improved patient prediction for a healthcare setting using the patient prediction system as described or otherwise envisioned herein, this novel patient prediction system has an enormous positive effect on patient care compared to prior art systems.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method for predicting simulated patient admissions and patient care pathways for a healthcare environment, comprising:
receiving, from an electronic health record database, healthcare records for a plurality of patients, wherein the plurality of patients comprises a plurality of different patient types;
adapting the received healthcare records to a common data format;
parameterizing the adapted healthcare records to generate a plurality of patient parameters, the plurality of patient parameters comprising for each patient a day of the week admission parameter, a time of day admission parameter, and a patient type parameter;
generating, from the plurality of patient parameters, a length of stay parameter for each of the plurality of different patient types;
generating, from the plurality of patient parameters, a transition probability for each of the plurality of different patient types, wherein the transition probabilities are stratified by one or more of likely day of transition and likely hour of transition;
predicting, for a time period in the healthcare environment, patient admissions using the patient parameters, wherein the patient admissions comprises predicted numbers of admitted patients during the time period and predicted patient type for each of the admitted patients;
predicting, using the length of stay parameters and the transition probabilities, a care pathway for some or all of the predicted patient admissions during the time period; and
reporting, via a user interface, the predicted patient admissions and predicted care pathways.

2. The method of claim 1, wherein the predicted care pathway comprises a length of stay prediction and one or more predicted transitions for a patient.

3. The method of claim 1, wherein the method further comprises applying a dimensionality reduction to the patient type parameters.

4. The method of claim 3, wherein the dimensionality reduction comprises clustering the patient type parameters by a similarity factor.

5. The method of claim 3, wherein the dimensionality reduction comprises clustering the patient type parameters by ICD codes in the healthcare records.

6. The method of claim 1, wherein the patient type parameter comprises one or more of patient admission diagnosis, patient discharge diagnosis, patient reason for visit, and patient complaint.

7. The method of claim 1, further comprising repeating the step of predicting a care pathway for some or all of the predicted patient admissions for one or more subsequent time periods.

8. The method of claim 1, wherein reporting the predicted patient admissions and predicted care pathways comprises reporting a patient census for the healthcare environment for one or more time periods.

9. A system for predicting simulated patient admissions and patient care pathways for a healthcare environment, comprising:
an electronic health record database comprising healthcare records for a plurality of patients, wherein the plurality of patients comprises a plurality of different patient types;
a processor configured to: (i) adapt the received healthcare records to a common data format; (ii) parameterize the adapted healthcare records to generate a plurality of patient parameters, the plurality of patient parameters comprising for each patient a day of the week admission parameter, a time of day admission parameter, and a patient type parameter; (iii) generate, from the plurality of patient parameters, a length of stay parameter for each of the plurality of different patient types; (iv) generate, from the plurality of patient parameters, a transition probability for each of the plurality of different patient types, wherein the transition probabilities are stratified by one or more of likely day of transition and likely hour of transition; (v) predict, for a time period in the healthcare environment, patient admissions using the patient parameters, wherein the patient admissions comprises predicted numbers of admitted patients during the time period and predicted patient type for each of the admitted patients; and (vi) predict, using the length of stay parameters and the transition probabilities, a care pathway for some or all of the predicted patient admissions during the time period; and
a user interface configured to report the predicted patient admissions and predicted care pathways.

10. The system of claim 9, wherein the predicted care pathway comprises a length of stay prediction and one or more predicted transitions for a patient.

11. The system of claim 9, wherein the processor is further configured to apply a dimensionality reduction to the patient type parameters.

12. The system of claim 11, wherein the dimensionality reduction comprises clustering the patient type parameters by a similarity factor, or wherein the dimensionality reduction comprises clustering the patient type parameters by ICD codes in the healthcare records.

13. The system of claim 9, wherein the patient type parameter comprises one or more of patient admission diagnosis, patient discharge diagnosis, patient reason for visit, and patient complaint.

14. The system of claim 9, wherein the processor is further configured to predict a care pathway for some or all of the predicted patient admissions for one or more subsequent time periods.

15. The system of claim 9, wherein reporting the predicted patient admissions and predicted care pathways comprises reporting a patient census for the healthcare environment for one or more time periods.
